(19) United States
(12) Patent Application Publication
(10) Pub. No.: US 2015/0272621 A1
(43) Pub. Date: Oct. 1, 2015

(54) Methods for Isolating and Removing Tissue During a Female Patient's Laparoscopic Surgery

(71) Applicant: David Leslie Zisow, Baltimore, MD (US)

(72) Inventor: David Leslie Zisow, Baltimore, MD (US)

(21) Appl. No.: 14/674,894

(22) Filed: Mar. 31, 2015

Related U.S. Application Data
(62) Division of application No. 14/231,822, filed on Apr. 1, 2014.

(51) Int. Cl. A61B 17/42

(52) U.S. Cl. CPC A61B 17/42; A61B 2017/4216; A61B 2017/00287

(57) ABSTRACT

A method for better isolating and removing a large mass of excised tissue from a female patient's abdominal cavity during a laparoscopic surgery includes the steps of: (1) vaginally inserting a collection bag that includes: (a) a flexible and resilient ring adapted to enable: the ring to be distorted for vaginal insertion, a specified ring open area sized to allow the mass of tissue to pass through it, and the ring to be removed from the patient's abdominal cavity through a laparoscopic-surgery-appropriately-sized incision, (b) a sleeve having a flexible wall and open end that is affixed to the ring and a defined interior volume sized so as to allow the mass of tissue to reside in the sleeve, and (2) utilizing the collection bag to contain the mass of tissue.

### CROSS-REFERENCE TO RELATED APPLICATIONS

This a Divisional Patent Application and claims the benefit of U.S. patent application Ser. No. 14/231,822, filed Apr. 1, 2014 by the present inventor. The teachings of this application are incorporated herein by reference to the extent that they do not conflict with the teachings herein.

### BACKGROUND OF THE INVENTION

1. Field of the Invention

The present invention generally relates to surgical devices and methods for collecting large mass tissue specimens surgically removed from a female patient. More specifically, the invention is directed to methods and devices for isolating and removing a large mass of tissue through a small incision in a female patient's abdominal cavity during a laparoscopic surgical procedure like a hysterectomy.

2. Description of the Related Art

Laparoscopic surgery is typically performed through trocars, which have relatively small openings or access ports through them that provide access across the abdominal wall and into the abdominal cavity. In these surgeries, tissue disposed within the abdominal cavity is typically excised and removed from the body. However, the removal of such tissue often proves to be difficult for many reasons, including the limited confines inherent in such laparoscopic surgeries and surgical instrument limitations.

Also, such excised tissue may include an infected or cancerous mass or organ, as well as blood, bile and other liquids, all referred to herein as tissue, which may pose infection issues or other complications if it is not enclosed while it is being transported from the surgical site to outside the abdominal cavity. Thus, it is always desirable to enclose such tissue as soon as possible at the surgical site and to keep it enclosed during its transport out of the abdominal cavity.

A quick survey of the patent literature in this area reveals that it is replete with myriad and diverse tissue retrieval and enclosure devices. Many of these utilize relatively small, rolled-up or folded bags or pouches that are deployed and opened in the abdominal cavity where tissue is placed in them and then they are closed for retraction. See, for example, U.S. Pat. Nos. 8,652,147, 8,486,087, 8,409,112, 7,650,887, 6,409,733, 5,647,372, 2009/0043315, 2009/0192510 and 2008/0221588.

A hysterectomy is the surgical removal of the uterus and is the second most common type of major surgery performed on women of childbearing age. A myomectomy is another commonly performed gynecologic procedure to remove fibroids while preserving the uterus. Advances in laparoscopic surgical equipment have enabled surgeons to remove fibroids and/or the uterus and/or ovaries laparoscopically. However, because of the large mass of such structures, these surgeries can present significant tissue isolation, enclosure and removal challenges.

The surgeon in a laparoscopic hysterectomy or myomectomy will usually have to expend a considerable amount of time and effort dissecting the fibroid(s) or uterus to small enough pieces so that they can be removed through the relatively small size of the opening of a trocar or other abdominal incision site. To help with this task, the surgeon may use a surgical instrument known as a morcellator, which consists of a hollow cylinder that penetrates the abdominal wall and has, at its open end, sharp edges that are used, often with a grasper that pulls the tissue into the cylinder, to mechanically cut or pulverized tissue.

A concern has arisen that the methods and instruments currently being used in laparoscopic surgeries are not doing a sufficiently adequate job in isolating and enclosing, during its removal from the abdominal cavity, the large mass of tissue involved in such surgeries. Cases have been alleged wherein, during a laparoscopic procedure, a previously undiagnosed sarcoma has been, because it was not adequately enclosed after excision and during removal, seeded throughout the abdominal cavity and thereby worsening a patient's prognosis.

To minimize this risk, what are needed are new laparoscopic surgery methods and devices for better enclosing and removing the large masses of tissues that typically need to be removed in gynecologic surgery. The present invention seeks to provide such improved methods and devices.

### SUMMARY OF THE INVENTION

Recognizing the need for improved methods and devices for enclosing and removing the large masses of tissue that typically need to be removed in laparoscopic surgery, the present invention is generally directed to providing such improved methods and devices.

In a preferred embodiment, the present invention is a method for better isolating and removing a large mass of excised tissue from a female patient's abdominal cavity during a laparoscopic surgery includes the steps of: (1) vaginally inserting a collection bag that includes: (a) a flexible and resilient ring adapted to enable: the ring to be distorted for vaginal insertion, and a specified ring open area sized to allow the mass of tissue to pass through it, (b) a sleeve having a flexible wall and open end that is affixed to the ring and a defined interior volume sized so as to allow the mass of tissue to reside in the sleeve, and (2) utilizing the collection bag to contain the mass of tissue.

In a first variant of this embodiment, the flexible and resilient ring is constructed from materials of construction that are selected so as to enable the ring, upon being situated in the abdominal cavity, to return to essentially the same shape that the ring took prior to being deformed for the vaginal insertion and except for being within the confines of the abdominal cavity.

In a second variant of this embodiment, the ring's configuration is further adapted to enable the ring to be further distorted so it can be removed from the patient's abdominal cavity a laparoscopic-hysterectomy-appropriate incision in the abdominal wall, and the method then further includes the step of (3) removing the collection bag through this laparoscopic-hysterectomy-appropriate incision in the abdominal wall of the patient.

In a third variant of this embodiment, the collection bag's sleeve is fabricated from materials of construction that are selected so as to enable the sleeve to continue to enclose and isolate a large tissue mass despite the stresses imposed on the sleeve by reducing the mass into smaller parts that can pass through the laparoscopic-surgery-appropriate incision in the abdominal wall of the patient, and the method then further includes the step of (4) while a large tissue mass is isolated and enclosed by the collection bag, reducing the mass into smaller parts that pass through the laparoscopic-hysterectomy-appropriate incision.

In a fourth variant of this embodiment, when said laparoscopic surgery is: (a) a total hysterectomy, the vaginal insertion of the collection bag traverses the open vaginal cuff apex, and (b) chosen from the group including a subtotal hysterectomy, a myomectomy, or the excision of a large adnexal mass, the vaginal insertion of the collection bag traverses an apical colpotomy incision into the pouch of Douglas.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1 is an illustrative depiction that shows the collection bag of the present invention having been vaginally inserted into a female patient's abdominal cavity and at a point in time during a laparoscopic surgery when the bag is being used to isolate and enclose a large mass of excised uterine tissue.

FIG. 2 is an illustrative depiction that shows the collection bag of the present invention having been vaginally inserted into a female patient's abdominal cavity and at a point in time during a laparoscopic surgery when the ring of the bag is beginning to be removed from the abdominal cavity though a wound retractor that lines a small incision in the patient's abdominal wall.

FIG. 3 is an illustrative depiction that shows the collection bag of the present invention having been vaginally inserted into a female patient's abdominal cavity and at a point in time during a laparoscopic surgery when the ring of the bag has been removed from the abdominal cavity but the bottom of the collection bag is still within the abdominal cavity and encloses a large mass of excised uterine tissue.

FIG. 4 shows a perspective view of a preferred embodiment of the bag of the present invention.

### DESCRIPTION OF THE PREFERRED EMBODIMENT

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The present invention generally relates to tissue isolation and removal methods and devices that can better isolate an excised, large uterine mass 1 during its removal from the abdominal cavity during a laparoscopic surgery. Since the surgical procedures involved in a laparoscopic surgery are well known in the art, they will not be discussed in detail herein. Instead, only the novel steps and devices of the present invention and how they differ from those utilized in a conventional laparoscopic surgery will be discussed in detail.

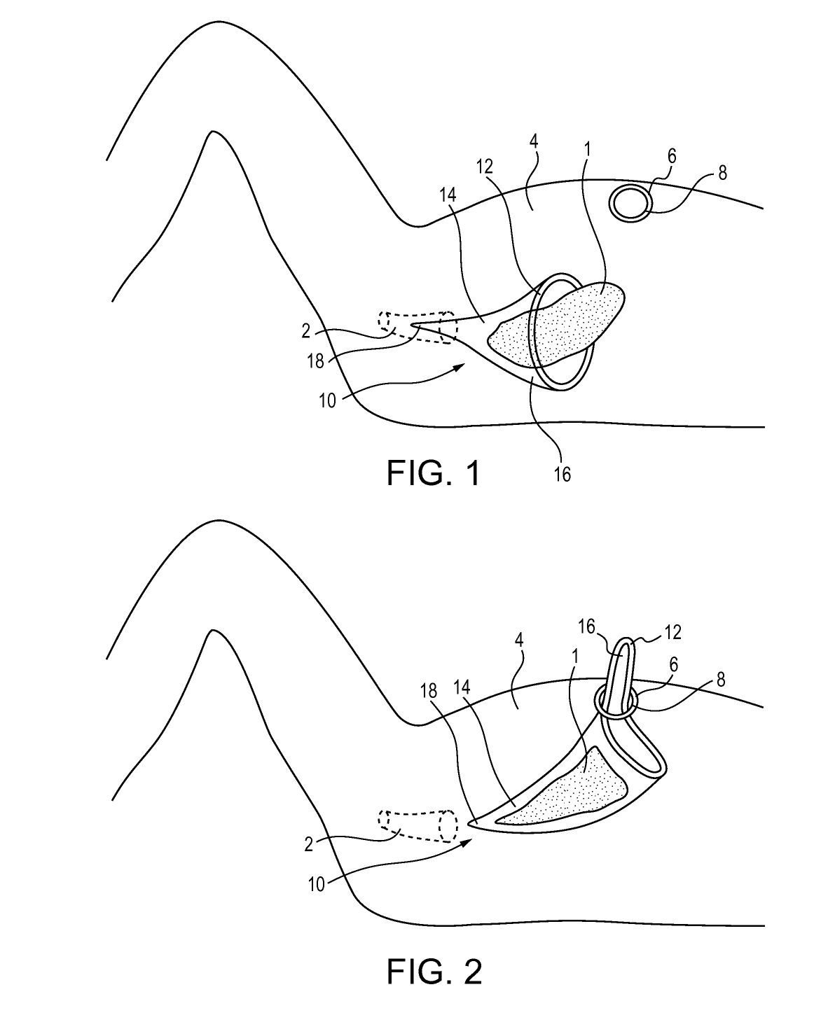

For example, FIG. 1 presents an illustrative depiction of a point in time during a laparoscopic surgery that is soon after a uterine excision and before the site of the excision has been closed. An especially-designed, tissue collection bag 10, which has a flexible and resilient opening ring 12, is seen to be introduced through the patient's vaginal canal 2 and into the abdominal cavity 4 where it is used to immediately enclose and isolate the large mass of tissue associated with the uterine excision. The resilience of the bag's ring allows it to quickly open and return to essentially the shape that it took prior to being deformed for its vaginal insertion.

In the case of total hysterectomy, the bag's insertion traverses the open vaginal cuff apex. In the case of a subtotal hysterectomy, a myomectomy, or excision of a large adnexal mass, the bag's insertion traverses an apical colpotomy incision into the pouch of Douglas.

This novel step in a laparoscopic surgery that has many advantages. The bag's large, distortable and resilient opening ring 12 and its large enclosed, interior volume make it much easier for the surgeon to quickly get the large mass of excised tissue into this bag 10 and to therefore isolate it from the rest of the abdominal cavity. The surgeon is aided in this task by the fact that this large volume bag 10 is, due to its vaginal insertion, being pulled up from below the surgical site and the excised tissue. Consequently, a surgeon can get this excised tissue isolated quicker than the surgeon has ever been able to do so before using conventional laparoscopic surgery techniques.

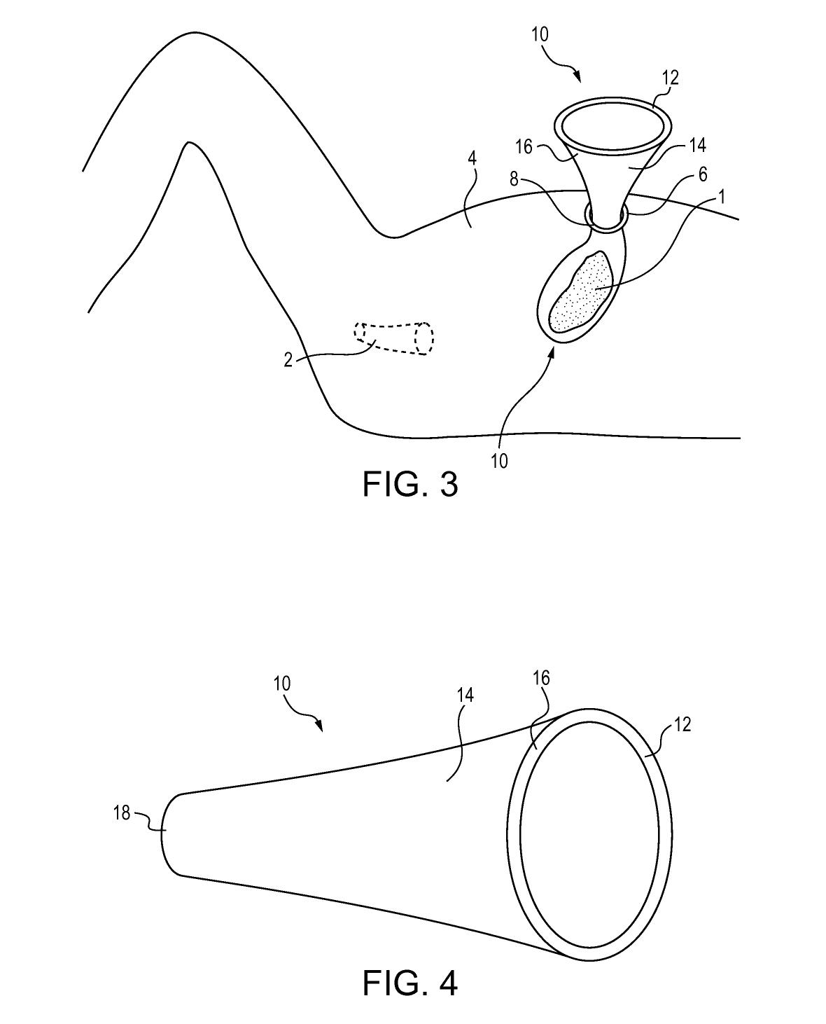

The configuration of these bags 10 have been adapted so that they allow any dissection or morcellation necessary for transport of the large mass out of the body to be performed within the isolated, large interior volume of the bag. The preparation for such processes is shown in FIGS. 2-3.

FIG. 2 shows an illustrative depiction of a point in time of a laparoscopic surgery during which a part of the large area, opening ring 12 of the bag of the present invention has been distorted and a portion of it has been removed from the patient through a wound retractor 6 that is being used at an abdominal laparoscopic incision site 8. FIG. 3 then depicts the point in time when the bag's opening ring 12 has been totally removed from the patient and has returned to its initial, undistorted shape while the majority of the sleeve 14 that extends beneath it is still within the patient's abdominal cavity.

This situation is novel and unlike anything comparable in a conventional laparoscopic surgery since the excised uterine tissue is now quickly and totally isolated by its enveloping bag 10 before any dissection or morcellation of this large mass is initiated so as to reduce it to a size that will allow this tissue to be removed through the laparoscopic surgery's small abdominal incision.

FIG. 4 shows a perspective view of a preferred embodiment of the bag 10 of the present invention. It is seen to include a flexible and resilient ring 12 that is deformable in order to enable it to be passed both through a patient's vaginal canal and a laparoscopic surgery incision in the patient's abdominal wall. This ring is initially released from its deformed shaped after it has passed through the uterine excision site and has reached the abdominal cavity. The overall shape of the ring can be circular, oval, elliptical or otherwise shaped to provide easy insertion through the vaginal canal and removal through a laparoscopic surgery incision or opening in the abdominal wall. Likewise, the cross-sectional shape through the ring 12 can be of any particular shape (e.g., round, elliptical, square, rectangular), and its materials of construction are selected so as to enhance the ring's ability to be flexible, resilient and durable.

A flexible walled, sleeve 14 made from a thin, tough, transparent, tissue- and bodily-fluids-impenetrable material (e.g., vinyl) includes an open proximal end 16 that is attached to the perimeter or circumference of the ring 12 and a closed distal end 18. There are many ways known in the art to attach the open end of the sleeve to the ring and therefore these will not be discussed herein. The sleeve has an area of its open, proximal end and a length between its ends that provides the bag 10 with an interior volume that is sized so as to accommodate the expected large mass of excised tissue.

Special care should be taken in selecting the material of construction for this bag as its material must be capable of continuing to enclose and isolate the excised tissue despite the stresses imposed on it by reducing within the bag the tissue into smaller parts that can pass through the typical, small-sized, laparoscopic surgery incisions.

For a typical laparoscopic surgery, a preferred embodiment of this bag may have dimensions in the range of: a ring diameter=15-23 cm, the area of a cross-sectional section of the ring=0.3-1 cm², the length of the bag=25-40 cm, and the volume of the bag=1,500-10,000 cm³. Bags with dimensions in these ranges can easily isolate and contain tissue masses in the range of 500-2,000 gms and larger.

The foregoing is considered as illustrative only of the principles of the present invention. Further, since numerous modifications and changes will readily occur to those skilled in the art, it is not desired to limit the invention to the exact construction and operation shown and described herein. Accordingly, all suitable modifications and equivalents may be resorted to, falling within the scope of the invention that is hereafter set forth in the claims to the invention.

I claim:

1. A method for better isolating and removing a large mass of excised tissue from a female patient's abdominal cavity during a laparoscopic surgery, said method comprising the steps of:
vaginally inserting a collection bag into the abdominal cavity of said patient, wherein said collection bag comprising:
(a) a flexible and resilient ring having a circumference that encloses a specified ring open area, said ring having a configuration adapted to enable: (i) said ring to be distorted so that said entire ring can be vaginally inserted to gain access for said entire collection bag into the abdominal cavity of said female patient, and (ii) the sizing of said specified ring open area is such that said large mass of tissue can pass though said ring open area, (b) a sleeve having a flexible wall and open and closed ends between which said wall encloses and defines the interior volume of said sleeve, wherein said sleeve open end is affixed to said ring, and said sleeve interior volume is sized so as to allow said large mass of tissue, after passing though said ring open area, to reside within and be isolated in the said sleeve, and
utilizing said collection bag to isolate and contain said large mass of tissue.

2. The method recited in claim 1, wherein:
said ring constructed from materials of construction that are selected so as to enable said ring, upon being situated in said abdominal cavity, to return to essentially the same shape that said ring took prior to being deformed for said vaginal insertion.

3. The method recited in claim 1, further comprising the step of:
removing said collection bag from the abdominal cavity of said female patient through a laparoscopic-hysterectomy-appropriate incision in the abdominal wall of said abdominal cavity,
wherein said ring configuration further adapted to allow for said collection bag removal through said laparoscopic-hysterectomy-appropriate incision.

4. The method recited in claim 2, further comprising the step of:
removing said collection bag from the abdominal cavity of said female patient through a laparoscopic-hysterectomy-appropriate incision in the abdominal wall of said abdominal cavity,
wherein said ring configuration further adapted to allow for said collection bag removal through said laparoscopic-hysterectomy-appropriate incision.

5. The method recited in claim 3, further comprising the step of:
while said large tissue mass is isolated and enclosed by said collection bag, reducing said large tissue mass into smaller parts that pass through said laparoscopic-hysterectomy-appropriate incision in the abdominal wall of said abdominal cavity,
wherein said sleeve constructed from materials of construction that are selected so as to enable said sleeve to continue to enclose and isolate said large tissue mass despite the stresses imposed on said sleeve by reducing said large tissue mass into said smaller parts.

6. The method recited in claim 4, further comprising the step of:
while said large tissue mass is isolated and enclosed by said collection bag, reducing said large tissue mass into smaller parts that pass through said laparoscopic-hysterectomy-appropriate incision in the abdominal wall of said abdominal cavity,
wherein said sleeve constructed from materials of construction that are selected so as to enable said sleeve to continue to enclose and isolate said large tissue mass despite the stresses imposed on said sleeve by reducing said large tissue mass into said smaller parts.

7. The method recited in claim 1, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

8. The method recited in claim 2, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

9. The method recited in claim 3, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

10. The method recited in claim 4, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

11. The method recited in claim 5, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

12. The method recited in claim 6, further comprising the step of:
when said laparoscopic surgery is a total hysterectomy that temporarily creates an open vaginal cuff apex, passing said collection bag, as part of said the vaginal insertion, through said open vaginal cuff apex, and
when said laparoscopic surgery is chosen from the group including a subtotal hysterectomy, a myomectomy, or the excision of a large adnexal mass that creates an apical colpotomy incision into the pouch of Douglas, passing said collection bag, as part of said the vaginal insertion, through said apical colpotomy incision into the pouch of Douglas.

13. The method recited in claim 2, wherein:
to provide for said vaginal insertion of said ring, said ring is dimensioned such that the cross-sectional section through said ring has a cross-sectional area in the range of 0.3-1 cm².

14. The method recited in claim 4, wherein:
to provide for said vaginal insertion of said ring, said ring is dimensioned such that the cross-sectional section through said ring has a cross-sectional area in the range of 0.3-1 cm².

15. The method recited in claim 6, wherein:
to provide for said vaginal insertion of said ring, said ring is dimensioned such that the cross-sectional section through said ring has a cross-sectional area in the range of 0.3-1 cm².

16. The method recited in claim 12, wherein:
to provide for said vaginal insertion of said ring, said ring is dimensioned such that the cross-sectional section through said ring has a cross-sectional area in the range of 0.3-1 cm².

17. The method recited in claim 13, wherein:
to further provide for said vaginal insertion of said ring, said ring is further dimensioned such that the circumference of said specified ring open area when said ring has assumed a circular shape is characterized by having an effective ring diameter in the range of 15-23 cm.

18. The method recited in claim 14, wherein:
to further provide for said vaginal insertion of said ring, said ring is further dimensioned such that the circumference of said specified ring open area when said ring has assumed a circular shape is characterized by having an effective ring diameter in the range of 15-23 cm.

19. The method recited in claim 15, wherein:
to further provide for said vaginal insertion of said ring, said ring is further dimensioned such that the circumference of said specified ring open area when said ring has assumed a circular shape is characterized by having an effective ring diameter in the range of 15-23 cm.

20. The method recited in claim 16, wherein:
to further provide for said vaginal insertion of said ring, said ring is further dimensioned such that the circumference of said specified ring open area when said ring has assumed a circular shape is characterized by having an effective ring diameter in the range of 15-23 cm.
